(19) United States
(12) Patent Application Publication
(10) Pub. No.: US 2014/0088675 A1
(43) Pub. Date: Mar. 27, 2014

(54) MEDICAL IMPLANTABLE LEAD AND MANUFACTURE THEREOF

(71) Applicant: COMPOSITE MATERIALS TECHNOLOGY, INC., SHREWSBURY, MA (US)

(72) Inventor: James Wong, Shrewsbury, MA (US)

(73) Assignee: COMPOSITE MATERIALS TECHNOLOGY, INC., SHREWSBURY, MA (US)

(21) Appl. No.: 14/030,840

(22) Filed: Sep. 18, 2013

Related U.S. Application Data
(60) Provisional application No. 61/704,322, filed on Sep. 21, 2012.

(51) Int. Cl. A61N 1/05

(52) U.S. Cl. CPC A61N 1/0587
USPC 607/119; 29/876

(57) ABSTRACT

A medical implantable lead comprising a core formed of a conductive wire formed from a biocompatible, corrosion-resistant conductive material, wrapped in a fibrous material formed of a valve metal, and surrounded by a biocompatible insulation material.

### CROSS REFERENCE TO RELATED APPLICATION

This application claims priority from U.S. Provisional Application Ser. No. 61/704,322, filed Sep. 21, 2012, the contents of which are incorporated herein by reference.

### BACKGROUND

The present invention relates to medical electrode leads and methods of manufacture thereof. The invention has particular utility in connection with cardiac pacing and defibrillation leads, i.e. suitable for intercardial stimulation of the heart with the help of an implantable pacemaker, or defibrillator, and will be described in connection with such utility, although other utilities are contemplated.

Surgically implanted cardiac devices play an important role in the treatment of heart disease. In the 50 years since the first pacemaker was implanted, technology has improved dramatically, and these devices have saved or improved the quality of countless lives. Pacemakers treat slow heart rhythms by increasing the heart rate or by coordinating the heart's contraction for some heart failure patients. Implantable cardioverter defibrillators stop dangerous rapid heart rhythms by delivering an electric shock. As the range of applications widens, the number of patients with cardiac devices continues to increase. Approximately 400,000 devices are implanted each year in the United States, and there are >3 million patients with implanted cardiac devices currently living in the United States.

Surgically implanted cardiac devices comprise two main parts, the pulse generator, a metal package that contains electric circuits and a battery, which usually is placed under the skin or on the chest beneath the collarbone, and the wires, or leads, which run between the pulse generator and the heart. In a pacemaker, these leads allow the device to control the heart rate by delivering small busts of electric energy. In a defibrillator, the leads allow the device to deliver a high-energy shock and convert dangerous rapid rhythms (ventricular tachycardia or fibrillation) back to a normal rhythm.

Although leads are designed to be implanted permanently in the body, occasionally leads fail due, for example, to a break in the insulation. In fact, in the last several years, two major manufacturers, Medtronic and St. Jude have recalled cardiac leads due to insulation failure or short circuit.

Cardiac leads typically are formed of concentrically stranded small via wires 2 formed from biocompatible, corrosion resistant, conductive materials such as MP 35 N, a cobalt based alloy having a nominal composition of 35% Ni, 35% Co, 20% Cr and 10% Mo around a conductive biocompatible, corrosion-resistant core 4 formed of, e.g. silver (FIG. 1). A typical lead is constructed with seven strands of MP35 N at 0.005 inches (127 microns) in diameter, cabled, and then formed into a helical spring shape hollow cable as shown in FIG. 2. This cable assembly is then electrically insulated with a suitable dielectric material 6 such as polyethylene or silicone rubber. Other cobalt-chromium alloys also have been used. The insulation also must be biocompatible and corrosion resistant. Most important, the insulation must be flexible and abrasion resistant in order to survive the tens of millions of flexure cycles the leads would be exposed to over the lifetime of a patient.

Failure of prior art cardiac leads typically is as a result of failure of the insulation. That is to say, while the conductive wires or fibers typically do not fracture, through continuous flexture and bending especially at tight bends and turns, the conductive wires or sheath material would sometimes wear and break through the insulation. As reported in the New York Times, Business Day Section, Wednesday, Aug. 22, 2012, Page B1; the lead failure was due to “inside out” abrasion where the wires had pushed through from the inside. Prior attempts to address the break-through problem, i.e., in terms of improving insulation composition and insulation thickness, have not proved to be entirely satisfactory. Making the insulation thicker will make the lead more robust; however, making the insulation thicker compromises flexibility which may present problems to the surgeon during implantation. Also, the leads must be sufficiently flexible once implanted so as to not exert stress on or injure body parts surrounding the implanted electrode.

### SUMMARY OF THE INVENTION

The present invention overcomes the aforesaid and other problems of the prior art, by wrapping an intermediate fibrous layer, formed e.g., of a biocompatible valve metal over the lead placed between the conductive cable and the insulating layer. Other fibrous material such as high strength polymers such as Kevlar or the like, or carbon fibers could also be used but the valve metal is the preferable choice. The intermediate layer which preferably is in the form of a braided fibrous material provides a cushioning and a lubricity which reduces insulation wear, and thus reduces lead failure.

The intermediate fibrous layer of biocompatible valve metal material may be formed quite thin, typically 2 to 50 microns in thickness, preferable 10 to 25 microns, most preferably about 5 to 10 microns in thickness.

The preferred valve metal comprises tantalum, although other valve metals such as niobium, titanium and zirconium which are also biocompatible, advantageously may be used in accordance with the present invention.

The fibrous valve metal material is formed following the teachings of my prior PCT Application Nos. PCT/US07/79249 and PCT/US08/86460, or my prior U.S. Pat. Nos. 7,480,978 and 7,146,709.

The process starts with fabrication of valve metal coated wire or filaments, by combining shaped elements of tantalum with a ductile material such as copper to form a billet. The billet is then sealed in an extrusion can, and extruded and drawn following the teachings of my aforesaid PCT applications and aforesaid U.S. patents.

The drawn wire is then braided on top of the hollow lead cable as shown in FIG. 2 and etched in HNO₃-H₂0 and to completely remove all the copper. The insulation layer can then be added.

### BRIEF DESCRIPTION OF THE DRAWINGS

Further features and advantages of the present invention will be seen from the following detailed description taken in conjunction with the accompanying drawings, wherein:

FIG. 1 diagram of MP35 N wire including a silver core;

FIG. 2 shows the spiral helical cable lead;

FIG. 3 diagramatically shows the overall process of the present invention; and

FIG. 4 shows an insulated, wrapped lead in accordance with the present invention.

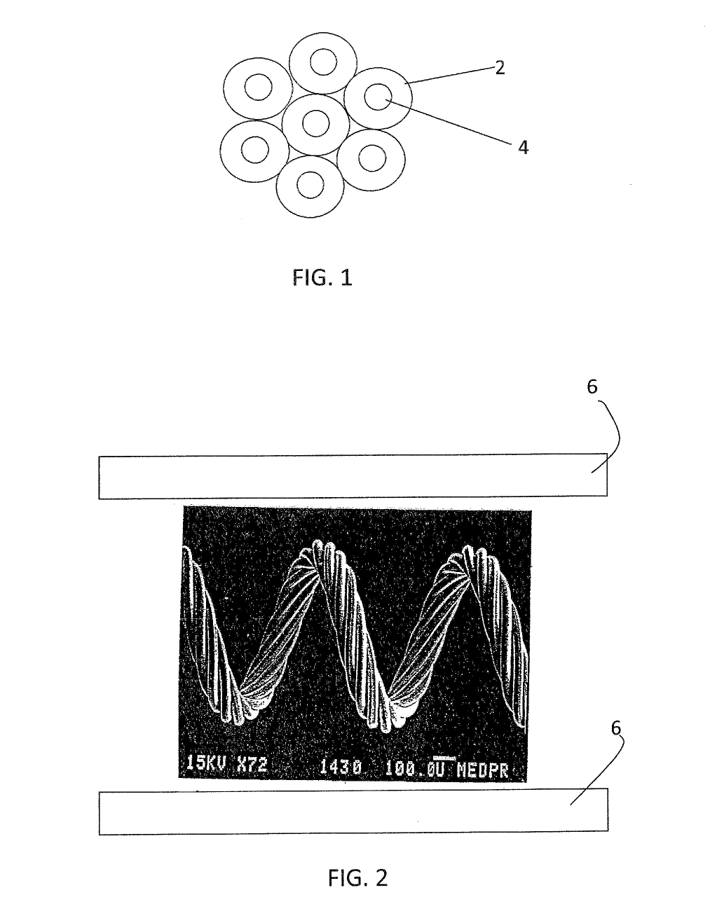

### DETAILED DESCRIPTION

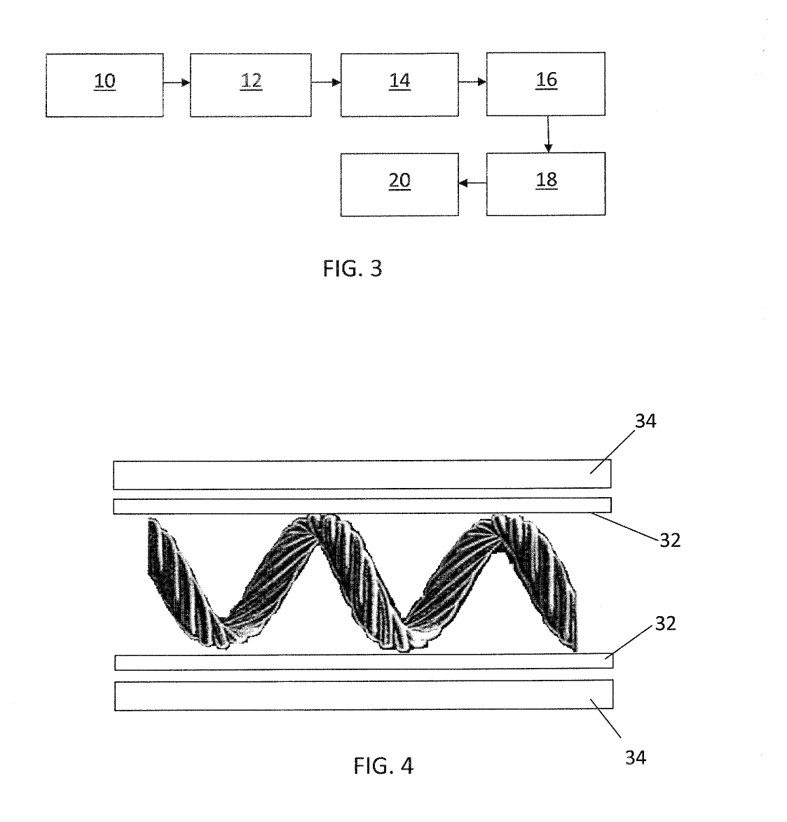

Referring to FIG. 3, the process starts with the fabrication of valve metal filaments, such as tantalum, by combining shaped elements of tantalum with a ductile material, such as copper to form a billet at step 10. The billet is then sealed in an extrusion can in step 12, and extruded and drawn in step 14 following the teachings of my prior PCT Applications No. PCT/US07/79249 and PCT/US08/86460, or my prior U.S. Pat. Nos. 7,480,978 and 7,146,709.

The filaments are formed into braids in step 16, which are then loosely wrapped around a conventional spiral helical cable lead in step 18, and the copper is removed by etching at step 20. An insulation layer is then formed over the wrapped cable in step 22.

Referring to FIG. 4, the resulting cable comprises a strand silver core wire, spiral wound cable, FIG. 2, formed of a loosely wrapped layer 32 of tantalum fibers, and surrounded by insulation 34.

A major factor contributing to the insulation failure is due to the design of the hollow lead cable. As can be seen in FIG. 2, the outer strands in contact with the insulation is extensively corrugated and moreover its orientation is transverse to the axis of the cable. So rather than sliding, the insulation is abraded, by the multiple exposed strands of the hard MP 35N metal wires and excessive wear can occur at tight bends and turns.

A feature and advantage of the present invention is that the tantalum fiber wrap is smooth, relatively soft ductile sleeve that provides a cushion and a lubricity which reduces pressure and rubbing on the insulation from the underlying wire as the lead is flexed. In other words, the wire and the insulation are permitted to slide relative to one another, thus reducing wear and a potential for insulation breakthrough.

The present invention provides significant improvements over prior art medical implantable leads by providing metallic fiber braid surrounding the metallic lead cable resulting in an extremely flexible lead, and which eliminates the abrasion and wear situation that exist with current medical implantable leads.

The invention claimed is:

1. A medical implantable lead comprising a core formed of a conductive wire formed from a biocompatible, corrosion-resistant conductive material, wrapped in a fibrous material, and surrounded by a biocompatible insulation material.

2. The lead of claim 1, wherein the fibrous material is selected from the group consisting of a valve metal, carbon, Kevlar and the like.

3. The lead of claim 1, wherein the valve material is selected from the group consisting of titanium, niobium, tantalum and zirconium.

4. The lead of claim 1, wherein the core comprises a metal core surrounded by a stranded metal cable.

5. The lead of claim 4, wherein the metal core comprises silver.

6. The lead of claim 4, wherein the stranded metal cable comprises a cobalt-chromium alloy material.

7. The lead of claim 1, wherein the valve metal fibrous material has a thickness of 2-50 microns.

8. The wire of claim 1, wherein the valve metal fibrous layer has a thickness of 10-25 microns.

9. The wire of claim 1, wherein the valve metal fibrous layer has a thickness of 5-10 microns.

10. A medical implantable lead comprising a cable formed of a biocompatible core metal, a wrap formed of a fibrous material surrounding the cable, and an insulating layer surrounding the wrap.

11. The medical implantable lead of claim 10, wherein the fibrous material is selected from the group consisting of valve metal, carbon and Kevlar or the like.

12. The medical implantable lead of claim 11, wherein the valve metal is selected from the group consisting of titanium, niobium, tantalum and zirconium.

13. A method for forming a medical implantable lead comprising providing a lead cable formed of a biocompatible, corrosion-resistant conductive metal, wrapping the core with a biocompatible, fibrous material, and encasing the resulting wrapped structure with an electrical insulation layer.

14. The method of claim 12, wherein the fibrous material is selected from the group consisting of a valve metal, carbon, Kevlar and the like.

15. The method of claim 14, wherein the valve metal is selected from the group consisting of titanium, niobium, tantalum and zirconium.
